(19) United States
(12) Patent Application Publication
(10) Pub. No.: US 2019/0002407 A1
(43) Pub. Date: Jan. 3, 2019

(54) PROCESS FOR THE PREPARATION OF CHIRAL 3-AMINO-PIPERIDINS, USEFUL INTERMEDIATES FOR THE PREPARATION OF TOFACITINIB

(71) Applicant: F.I.S. - Fabbrica Italiana Sintetici S.p.A., Montecchio Maggiore (VI) (IT)

(72) Inventors: Mireia Pastó Aguilà, Barcelona (ES); Sara Preciado Gallego, Barcelona (ES); Emanuele Miserazzi, Bologna (ES)

(73) Assignee: F.I.S. - Fabbrica Italiana Sintetici S.p.A., Montecchio Maggiore (VI) (IT)

(21) Appl. No.: 16/017,390

(22) Filed: Jun. 25, 2018

(30) Foreign Application Priority Data
Jun. 29, 2017 (EP) 17178755.9

(51) Int. Cl. C07D 211/56; C07D 487/04; B01J 31/22

(52) U.S. Cl. CPC C07D 211/56; C07D 487/04; B01J 31/2291; B01J 2231/645; B01J 2531/822; B01J 2531/842; B01J 31/2295

(57) ABSTRACT

Object of the present invention is an improved process for the preparation of (3R,4R)-1-benzyl-4-methylpiperidin-3-amine by means of chiral Rhodium catalysts.

### CROSS-REFERENCE TO RELATED APPLICATIONS

This application claims benefit to European Patent Application No. EP17178755.9, filed Jun. 29, 2017, the entire contents of which are incorporated by reference herein as if fully set forth.

### TECHNICAL FIELD

The object of the present invention is an improved process for the synthesis of a key intermediate of the pharmaceutically active substance known as Tofacitinib or salt thereof.

### BACKGROUND

3-amino-piperidine compounds represent valuable intermediates for the preparation of pharmaceutically active agents. For example, the Janus kinase 3 (JAK3) inhibitor named Tofacitinib, having the structural formula (I):

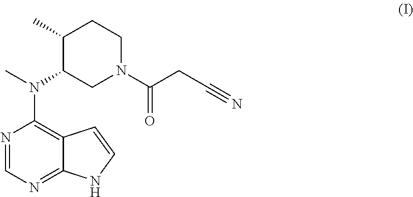 [SMILES: C[C@@H]1CCN(C(=O)CC#N)C[C@@H]1N(C)c1ncnc2[nH]ccc12]

wherein both the asymmetric carbons have R configuration; comprises a 4-methyl-3-(methylamino)piperidin-1-yl moiety having the structural formula

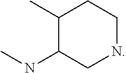

Janus kinase 3 (JAK3) inhibitors are a group of compounds that are classified to interfere with the Janus kinase signal transducer and activator of transcription (JAK-STAT) signalling pathway transmitting extracellular information into the cell nucleus and influencing DNA transcription.

Tofacitinib as one JAK3 inhibitor was found to be effective for many applications and can be used against e.g. rheumatoid arthritis, psoriasis, inflammatory bowel disease and other immunological diseases, as well as for prevention of organ transplant rejection.

Hu, et al., Org. Lett. 2002, 4, pages from 4499 to 4502, describes a synthetic route for preparation of (3S)-amino-piperidine intermediates. In this synthetic route, predominantly products having trans-configuration of the substituents in 3 and 4 position of the piperidine ring are obtained. However, trans-configuration is not desired for intermediate compounds for preparing of Tofacitinib. Rather, cis-configuration is desired.

Brown, et al, Org. Proc. Res. Dev. 2003, 7, pages from 115 to 120, and in particular at last part of second column of page 119 describes the preparation of 3-(methylamino)-4-methylpiperidine building block via reductive amination of 4-methylpiperidin-3-one using methylamine as reagent. The ketone was prepared by a combined hydroboration/oxidation process of methyl-tetrahydropyridine as describes in Iorio, et. al., in Tetrahedron 1970, 26, page 5519 and Ripin, et al., Tetrahedron Lett. 2000, 41, page 5817. The resulting compound was subjected to oxidation by an excess of SO₃ pyridine complex. The process involves application of hazardous reagents in the form of hydroborating agents such as NaBH₄ or BH₃ complexes and strong oxidants such as hydrogen peroxide, bleach or Oxone®.

Hao, et al., Synthesis 2011, 8, pages 1208 to 1212, describes a synthetic route which starts from ethyl 1-benzyl-3-oxopiperidine-4-carboxylate hydrochloride. It is noteworthy to mention that the process is lengthy in terms of the amount of procedural steps required. Furthermore, the process requires hazardous and expensive reagents and starts from an advance intermediate. Asymmetric reduction of olefin in the presence of cobalt catalysts affords modest diastereomeric excess of 71%. Reductive amination to incorporate methyl group on amine part of molecule represents the key step, however, accomplishing this reductive amination is problematic. Besides, stereoselective transformation of ester group to methyl requires costly and hazardous reagents.

Furthermore, Cai. Et al.; Org. Proc. Res. Dev. 2005, 9, pages 51 to 56 (in particular pages 55 and 56) describes an alternative procedure, according to the following scheme 1, wherein a protected 3-amino-4-picoline is converted to 3-amino-piperidine by means of total reduction of the pyridine ring.

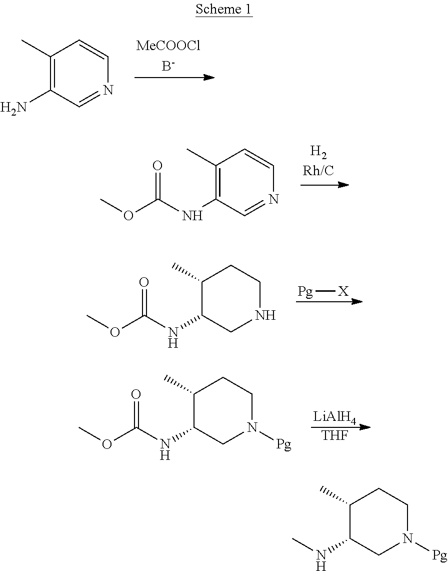

However, in this synthetic pathway, the rare and costly 3-amino-4-picoline is required as starting material. Besides, hydrogenation has to be carried out at high hydrogen pressure in order to achieve total reduction of the pyridine moiety to piperidine. The products of said synthesis are however racemic compounds.

WO 2007/012953, on example 3 at page 19, describes the last step of a further synthetic pathway in which 3-amino-4-picoline is used as starting material. As can be gathered from scheme 2, the pathway contains the steps of benzyl activation of pyridine ring and partial reduction using sodium borohydride. In the final step, asymmetric hydrogenation is carried out by means an optically active rhodium complex (formed by bis(1,5-cyclooctadiene)rhodium triflate and ferrocenyl phosphine Josiphos SL-J009-1™), in a mixture of tetrahydrofuran and ethanol to obtain (3R,4R)-(1-benzyl-4-methyl-piperidine-3-yl)-methylamine in 97% cis product in modest enantioselectivity of at best 66% e.e., and ratio Z/E, i.e. ratio between cis/trans diastereoisomers (abbreviated Dr), of 48.5.

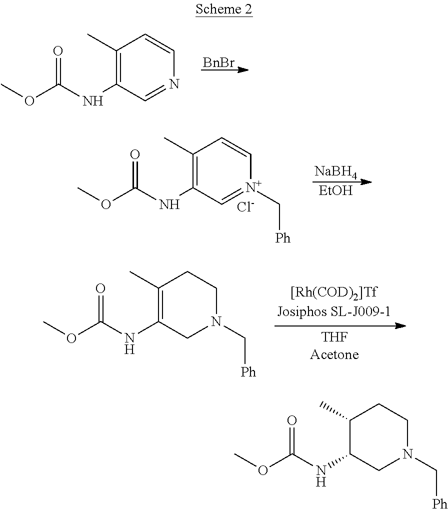

This potential prior art has not been confirmed by our experimentation. In particular, repeating the experiment 3 of WO 2007/012953, the (3S,4S)-(1-benzyl-4-methyl-piperidine-3-yl)-methylamine was isolated and not the alleged (3R,4R)-(1-benzyl-4-methyl-piperidine-3-yl)-methylamine as stated in said application, as confirmed by chiral HPLC analysis (see analytical method in the experimental part).

However, claims from 44 to 47 of the application WO 2007/012953 appears to disclose a process for the preparation of enantiomerically enriched piperidine of formula:

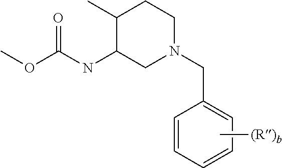

wherein R″ in chosen from the group consisting of hydrogen, (C1-C6)alkyl and CF3 groups, b is an integer from 0 to 4, by asymmetrically hydrogenation of a tetrahydropyridine of formula:

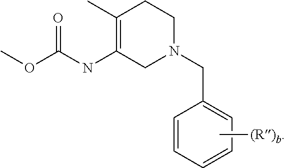

The object of the present invention is to provide an improved process for preparing 3-amino-piperidine compounds representing valuable key intermediates for the preparation of pharmaceutically active agents.

### SUMMARY

The problem addressed by the present invention is therefore that of providing an improved process for the preparation of a 3-(methylamino)-4-methylpiperidine moiety.

In particular, the problem of the present invention is to provide a better process for the preparation of a 3-(methylamino)-4-methylpiperidine moiety, with improvements especially in terms of enantiomeric excess (e.e.) and/or diastereomeric ratio and/or molar yield, or conversion.

This problem is solved by a process for the synthesis of 3-(methylamino)-4-methylpiperidine moiety as outlined in the annexed claims, whose definitions are integral part of the present description.

Further features and advantages of the process according to the invention will result from the description hereafter reported of examples of realization of the invention, provided as an indication of the invention.

### DETAILED DESCRIPTION OF THE PREFERRED EMBODIMENTS

The present invention provides a process for the preparation of a compound of formula (II) or a salt thereof:

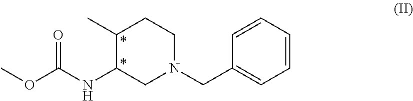 [SMILES: COC(=O)NC1CN(Cc2ccccc2)CCC1C]

wherein the substituents at positions 3 and 4 of the piperidine ring are in a cis configuration, i.e. both the asymmetric carbons have R configuration or S configuration.

The process comprising the asymmetrical hydrogenation of the compound of formula (III) or a salt thereof:

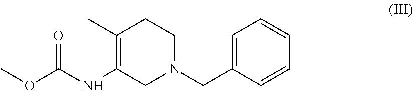 [SMILES: COC(=O)NC1=C(C)CCN(Cc2ccccc2)C1]

in a solvent, and the presence of a Rh(I) complex and of an optically active ferrocenyl phosphine.

In particular, the compound of formula (II) or a salt thereof has, at the asymmetric carbons marked with the symbol *, 3-R and 4-R optical configuration or 3-S and 4-S optical configuration or a mixture thereof, with the exclusion of the racemic mixture.

According to the invention, the solvent is 2,2,2-trifluoroethanol (TFE) or methanol, more preferably is TFE.

It has been indeed surprisingly found that said solvents allows the preparation of the compound (II) with enantiomeric excess (abbreviated e.e.) >67%, or at least 70%, and/or with very high conversions.

Furthermore, it has been indeed surprisingly found that the TFE solvent allows the preparation of the compound (II) with the completed conversion of the compound of formula (III) to the compound of formula (II), and with robust reproducibility of the results.

Moreover, it has been indeed surprisingly found that the TFE solvent allows the preparation of the compound (II) with a lower pressure and/or at lower temperature, and in much shorter reaction time.

The solvent can be used in amounts of at least 4 volumes, preferably at least 5 volumes, more preferably of between 8 and 12 volumes. Preferably, from 5 to 10 volumes of TFE or from 10 to 20 volumes of methanol are used.

The term “volume” referred to the solvent is to be understood as a volume per amount per weight amount of compound of formula (III).

The term “volume” thus means volume of solvent per unit of product, thus, for example, 1 volume is 1 Liter per 1 Kilo, or 1 mL for 1 gram, or 1 microliter per 1 milligram. Thus, 10 volumes means for example 10 liters per 1 Kilogram of substance.

The Rh(I) complex is a neutral complex of the general formula (IVa) or (IVb):

[RhLA]₂ (IVa)

[RhL₂A] (IVb)

wherein L represents a C₄₋₁₂ diene or two C₂₋₁₂ alkene molecules; A is chlorine, bromine, iodine, trifluoromethanesulfone, tetrafluoroborate or acetylacetonate. More preferably, L is norbornadiene or 1,5-cyclooctadiene and/or A is trifluoromethansulfone.

The optically active ferrocenyl phosphine is a compound of following formula (V):

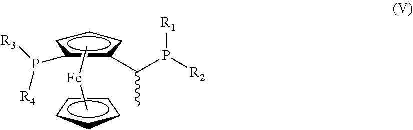

wherein R₁, R₂, R₃ and R₄ are independently selected between linear or branched C₁₋₅ alkyl, unsubstituted aryl, substituted aryl with a linear or branched C₁₋₅ alkyl group or is a cyclic C₅₋₆ alkyl.

In the optically active ferrocenyl phosphine of formula (V) of the process of the invention, the linear or branched C₁₋₅ alkyl of R₁, R₂, R₃ and R₄, can be methyl, ethyl, n-propyl, isopropyl, n-butyl, isobutyl, sec-butyl, tert-butyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl.

The unsubstituted aryl group of R₁, R₂, R₃ and R₄ can be phenyl, furyl or naphthyl.

In the substituted aryl with a linear or branched C₁₋₅ alkyl group of R₁, R₂, R₃ and R₄, the C₁₋₅ alkyl group is methylene, ethyl, n-propyl, isopropyl, n-butyl, isobutyl, sec-butyl, tert-butyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, methoxy, ethoxy, n-propoxy, isopropoxy, n-butoxy, sec-buthoxy, n-pentoxy, etc.

The cyclic C₅₋₆ alkyl group of R₁, R₂, R₃ and R₄ can be cyclopentyl or cyclohexyl.

In a preferred embodiment, the optically active ferrocenyl phosphine compound of formula (V):

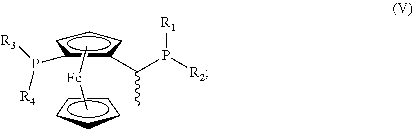

can be selected in a group comprising:

(R)-1-[(S_{P})-2-(Diphenylphosphino)ferrocenyl]ethyldi-tert-butyl phosphine with CAS [155830-69-6], is also sometimes named Josiphos SL-J002, having the following formula:

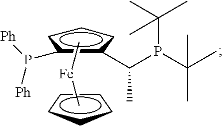

(S)-1-[(R_{P})-2-(Di-tert-butylphosphino)ferrocenyl] ethylbis (2-methyl phenyl)phosphine with CAS [849924-77-2], is also sometimes named Josiphos SL-J505-2, having the following formula:

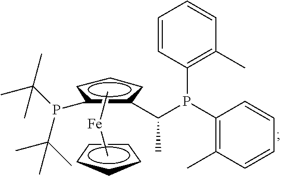

(S)-1-{(R_{P})-2-[Bis[4-(trifluoromethyl)phenyl]phosphino]ferrocenyl} ethyldi-tert-butylphosphine with CAS [849924-37-4], is also sometimes named Josiphos SL-J011-2, having the following formula:

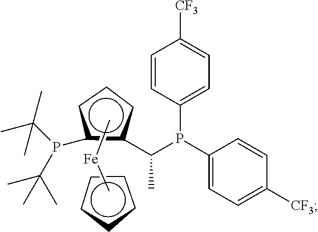

(S)-1-[(R_{P})-2-(Di-tert-butylphosphino)ferrocenyl]ethyldiphenyl phosphine with CAS [223121-01-5], is also sometimes named Josiphos SL-J502-2, having the following formula:

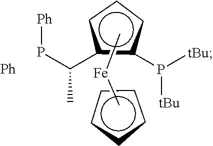

(R)-1-[(S_{P})-2-[Bis(4-methoxy-3,5-dimethylphenyl)phosphino]ferrocenyl}ethyldi-tert-butylphosphine with CAS [187733-50-2], is also sometimes named Josiphos SL-J013-1, having the following formula:

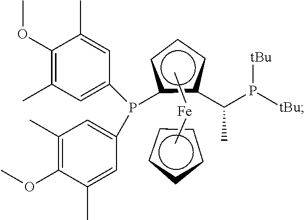

(S)-1-[(R_{P})-2-(diphenylphosphino)ferrocenyl]ethyldicyclohexyl phosphine with CAS [162291-02-3], is also sometimes named Josiphos SL-J001-2, having the following formula:

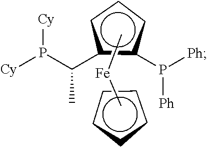

(S)-1-[(R_{P})-2-(dicyclohexylphosphino)ferrocenylethyl] diphenyl phosphine with CAS [162291-01-2], is also sometimes named Josiphos SL-J004-2, having the following formula:

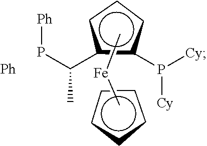

(R)-1-[(S_{P})-2-(Dicyclohexylphosphino)ferrocenyl]ethyldi-tert-butyl phosphine with CAS [158923-11-6], is also sometimes named Josiphos SL-J009-1, having the following formula:

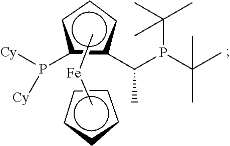

(R)-1-{(S_{P})-2-[Di(2-furyl)phosphino]ferrocenyl}ethyldi-tert-butyl phosphine with CAS [849924-41-0], is also sometimes named Josiphos SL-J212-1, having the following formula:

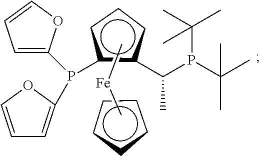

or their enantiomers.

More preferably, the ferrocenyl phosphine is (R)-1-[(S_{P})-2-(Di-tert-butylphosphino)ferrocenyl]ethylbis(2-methylphenyl) phosphine, having the formula (VI):

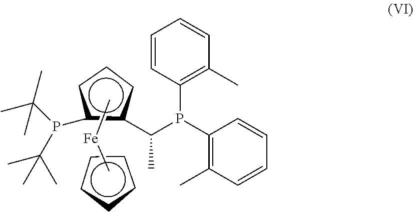

or its (S,R_{P}) enantiomer, since they provides the best results in terms of e.e.

It should be understood that, by using the (S,Rp)-enantiomer of the above ferrocenyl phosphine of formula (VI), the (3S,4S)-enantiomer of the compound of formula (II) is obtained, while the preferred (R,Sp)-enantiomer of the above ferrocenyl phosphine of formula (VI) gives the preferred opposite (3R,4R)-enantiomer of the compound of formula (II).

The rhodium (I) complex and the ferrocenyl phosphine are preferably used in a percent amount (w/w) of from about 0.5 to about 2%, more preferably about 1%, compared to the amount of compound of formula (III).

The term “about” in the whole context of the present invention means a variation of the indicated value of ±15%. For example, if “about 1%” of a compound is indicated, this means that an amount ranging from 0.85% to 1.15% can be used.

This reaction is preferably conducted at a temperature selected in the range of from 25° C. to 70° C., preferably from 30° C. to 60° C., and at a hydrogen pressure of from 2 and 20 bar, preferably from 3 to 15 bar. The reaction time is above 24 hours, preferably above 40 hours.

In particular, when TFE is used as the solvent, the reaction temperature is preferably selected in the range of from 30° C. to 60° C., while when methanol is used as the solvent, the reaction temperature is preferably selected in the range of from 50° C. to 70° C.

In particular, when TFE is used as the solvent, the reaction temperature is preferably selected in the range of from 30° C. to 60° C., more preferably in the range of from 30° C. to 40° C.

In a particularly preferred embodiment, when methanol is used as the solvent, the reaction temperature is preferably selected in the range of from 50° C. to 70° C., more preferably in the range of from 55° C. to 65° C., again more preferably at 60° C.

Moreover, preferably, when TFE is used as the solvent, the reaction pressure is selected in the range of from 2 to 15 bar, while when methanol is used as the solvent, the reaction pressure is selected in the range of from 10 to 20 bar.

In particular, when TFE is used as solvent, the reaction pressure is preferably selected in the range of from 2 to 15 bar, more preferably in the range from 2 to 5 bar, more preferably in the range of about 3 to about 4 bar, more preferably at about 3.5 bar.

In a particularly preferred embodiment, the inventive process comprises the hydrogenation of the compound of formula (III) to give the compound of formula (II), when TFE is used as solvent, is carried out at temperature in the range of from 30° C. to 60° C. and with a hydrogen pressure selected in the range from 2 to 5 bar.

Moreover, preferably, when TFE is used as the solvent, the inventive process comprises the hydrogenation of the compound of formula (III) to give the compound of formula (II), is carried out at temperature in the range of from 30° C. to 40° C. and with a hydrogen pressure selected in the range of about 3 to about 4 bar.

In a particularly preferred embodiment, the inventive process comprises the hydrogenation of the compound of formula (III) to give the compound of formula (II) in about 10 volumes of 2,2,2-trifluoroethanol, at temperature in the range of from 30° C. to 40° C. and with a hydrogen pressure selected in the range of about 3 to about 4 bar.

In a particularly preferred embodiment, the inventive process comprises the hydrogenation of the compound of formula (III) to give the compound of formula (II) in about 10 volumes of 2,2,2-trifluoroethanol, at temperature in the range of from 30° C. to 40° C. and with a hydrogen pressure selected in the range of about 3 to about 4 bar.

The reaction time to complete the reaction depending on the condition is 60 hours or less, e.g. 5 hours.

The process of the invention allows to obtain the compound of formula (II), either as a (3S,4S)- or (3R,4R) enantiomer, with an enantiomeric excess (e.e.) higher than 67%, preferably of at least 70%, more preferably of at least 75%.

The process of the invention may also comprise the following steps:

(i) reduction of the compound of formula (II), or salts thereof:

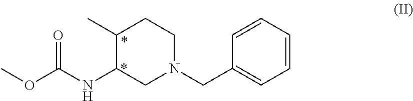 [SMILES: COC(=O)NC1CN(Cc2ccccc2)CCC1C]

wherein the asymmetric carbons marked with the symbol * have 3-R and 4-R optical configuration or 3-S and 4-S optical configuration or mixture thereof, with the exclusion of the racemic mixture; to give the compound 1-benzyl-N,4-dimethylpiperidin-3-amine of formula (VII) or salts thereof:

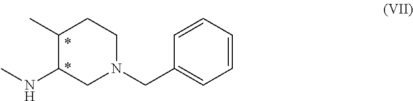 [SMILES: CNC1CN(Cc2ccccc2)CCC1C]

wherein the asymmetric carbons marked with the symbol * have 3-R and 4-R optical configuration or 3-S and 4-S optical configuration or mixture thereof, with the exclusion of the racemic mixture.

(ii) optionally, conversion of the compound of formula (VII), as the (R,R)-enantiomer, or salts thereof, of formula (VII-RR):

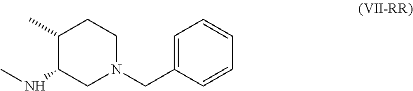 [SMILES: CN[C@H]1CN(Cc2ccccc2)CC[C@H]1C]

into Tofacitinib, or salts thereof, of formula (I):

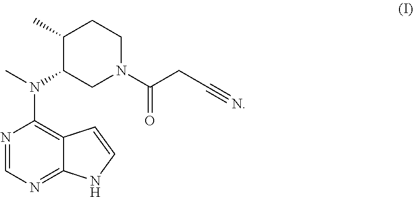 [SMILES: C[C@@H]1CCN(C(=O)CC#N)C[C@@H]1N(C)c1ncnc2[nH]ccc12]

Step (i) can be performed by reacting the compound of formula (II) with an hydride in a solvent. Preferably, lithium aluminum hydride is used. Preferred reaction conditions provide for the use of an excess of hydride of at least 3 equivalents in a THF solvent at reflux temperature.

This reaction can be followed by the salification of the compound of formula (VII), for example with hydrogen chloride in a non-aqueous solvent.

The salification of the compound of formula (VII) with hydrogen chloride generates the compound of formula (VIII):

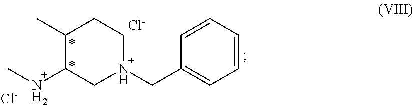 [SMILES: C[NH2+]C1C[NH+](Cc2ccccc2)CCC1C ; [Cl-] ; [Cl-]]

said bis-HCl salt of the compound (VII) wherein the asymmetric carbons marked with the symbol * have 3-R and 4-R optical configuration or 3-S and 4-S optical configuration or mixture thereof, allows an efficient purging of the undesired isomer, to obtain an efficient enrichment in terms of enantiomeric excess.

In particular, the compound (VIII) bis-HCl salt can be prepared in a solution of compound of formula (VII) in methanol by addition of hydrochloric acid. In particular, the addition of from 1% to 5% of water to the solution increases the enrichment in terms of enantiomeric excess.

Step (i) substantially retains the optical configuration of the starting compound of formula (II). This means that, if compound of formula (II) with an e.e. higher than 67% is reacted, a compound of formula (VII) with an e.e. higher than 67% will be obtained.

Step (ii) can be performed according to well-known methods, for example those described in WO 2014/195978 in example from 1 to 6 (page from 25 to page 27). The conversion of the compound of formula (VII), as the (R,R)-enantiomer, or salts thereof, into Tofacitinib, or salts thereof, of formula (I) also substantially retains the enantiomeric excess of the starting compound of formula (VII).

Moreover, the compound of formula (III) can also be prepared as described in the WO 2007/012953 (see example 2).

All of the intermediates and compounds of the present invention in particular those of formula (I), (II), (III), (VII) can be in isolated or in not isolated form, from the reaction mixture wherein they are prepared.

According to the preferred embodiment, all of the intermediates and compounds isolated are typically in form of a solid.

The following scheme shows the overall process for preparing the compound of formula (VII) and (VIII) having 3R and 4R configuration.

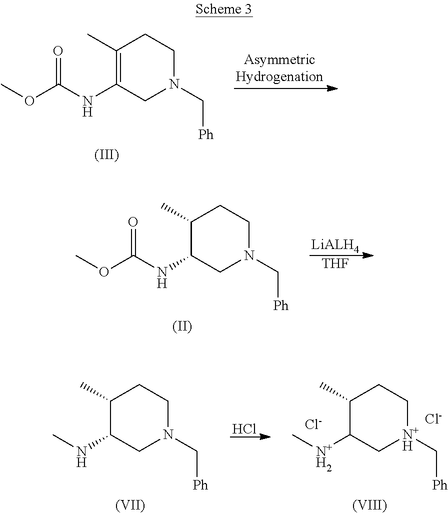

### Experimental Part

The Rh(I) complex and the ferrocenyl ligand, are reactants largely commercially available, for example, for supplied by: Sigma Aldrich (USA) or Alfa Aesar (Germany).

The starting material, i.e. the compound of formula (III) can be prepared according the teaching of international application publication No. WO 2007/012953 in the example 2 at pag. 19.

Asymmetric Hydrogenation.

### Example 1

### Preparation of methyl (1-benzyl-4-methylpiperidin-3-yl)carbamate (II)-in methanol

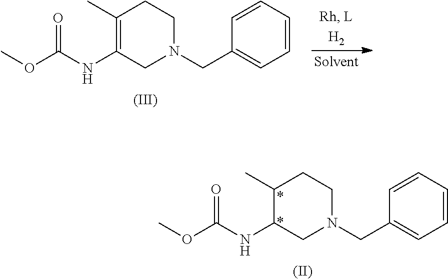

Six experiments have been carried out keeping constant the substrate concentration, temperature, catalyst and ligand loading and type, and solvent according to the conditions of the table below, as well as any other parameter/variable.

Conditions

| Rh(I) | Reaction | Ligand Type | Ligand |
|---|---|---|---|
| 0.01 eq | 60° C. | Josiphos SL- | 0.01 eq |

Procedure

To a 250 mL pressure vessel were added (1-benzyl-4-methyl-1,2,5,6-tetrahydropyidin-3-yl) carbamate (compound (III), 1.0 g, 3.84 mmol), bis(1,5-cyclooctadiene)rhodium(I) trifluoromethanesulfonate (18.0 mg, 0.0384 mmol, Aldrich 530840, CAS: 99326-34-8), (S)-1-[(Rp)-2-(di-tert-butylphosphino)ferrocenyl]ethylbis(2-methylphenyl)phosphine (22.0 mg, 0.0384 mmol, Aldrich 88756, CAS: 849924-77-2). The solids were purged with nitrogen (5×5 bar) then the methanol was added. The solution was purged with nitrogen (5×5 bar) followed by hydrogen. The reaction was heated and maintaining the hydrogen pressure. After the complete reaction, the mixture was cooled and purged with nitrogen (5×5 bar). An aliquot was removed for HPLC analysis to confirm full reaction conversion. The mixture was concentrated to dryness and methyl (1-benzyl-4-methylpiperidin-3-yl)carbamate (II) was isolated as an oil and analysed for chirality.

TABLE 1
Comparison of the condition, with same ligand.

| | | | | | | Dr | e.e. % |
|---|---|---|---|---|---|---|---|
| | Pressure | MeOH | Time | Conversion | Yield | (cis/ | (S,S |
| Trial | (bar) | (V) | (h) | (%) | (%) | trans) | isomer) |
| 1 | 15 | 20 | 72 | 100% | / | 51.3 | 72% |
| 2 | 15 | 20 | 48 | 82% | 71% | 50.33 | 72% |
| 3 | 15 | 10 | 48 | 97% | 52% | >300 | 74.8% |
| 4 | 15 | 15 | 72 | 76% | / | >100 | 72.8% |
| 5 | 3.5 | 10 | 24 | 100% | 89% | 49.4 | 70.8% |
| 6 | 3.5 | 10 | 24 | 100% | 99% | 42 | 73.2% |

Note:
Dr is the diastereoisomeric ratio between the sum of the amount of the cis enantiomers over the sum of the amount of two trans isomer.

In others words Dr=((3S,4S)+(3R,4R))/((3R,4S)+(3S,4R)).

### Example 2

### Preparation of methyl (1-benzyl-4-methylpiperidin-3-yl)carbamate (II)-in TFE

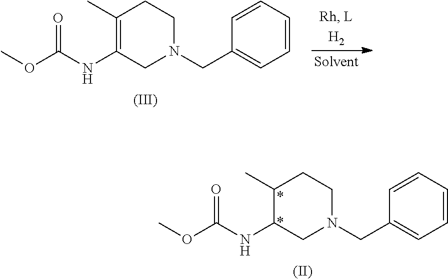

Ten experiments have been carried out keeping constant the substrate concentration, catalyst ligand loading and type, and solvent according to the conditions of the table below, as well as any other parameter/variable. For these examples TFE was chosen as the solvent.

Conditions

| Rh(I) | Ligand Type | Ligand eq |
|---|---|---|
| 0.01 eq | Josiphos SL-J505- | 0.01 eq |

Procedure

To a 250 mL pressure vessel were added (1-benzyl-4-methyl-1,2,5,6-tetrahydropyidin-3-yl) carbamate (compound (III), 1.0 g, 3.84 mmol), bis(1,5-cyclooctadiene)rhodium(I) trifluoromethanesulfonate (18.0 mg, 0.0384 mmol, Aldrich 530840, CAS: 99326-34-8), (S)-1-[(Rp)-2-(di-tert-butylphosphino)ferrocenyl]ethylbis(2-methylphenyl)phosphine (22.0 mg, 0.0384 mmol, Aldrich 88756, CAS: 849924-77-2). The solids were purged with nitrogen (5×5 bar) then the TFE was added. The solution was purged with nitrogen (5×5 bar) followed by hydrogen. The reaction was heated and maintaining the hydrogen pressure. After the complete reaction, the mixture was cooled and purged with nitrogen (5×5 bar). An aliquot was removed for HPLC analysis to confirm full reaction conversion. The mixture was concentrated to dryness and methyl (1-benzyl-4-methylpiperidin-3-yl)carbamate (II) was isolated as an oil and analysed for chirality.

TABLE 2
Comparison of the condition, with same ligand.

| | | | | | | | Dr | e.e. % |
|---|---|---|---|---|---|---|---|---|
| | Pres. | Temp. | TFE | Time | Conv. | Yield | (cis/ | (S,S |
| Trial | (bar) | (° C.) | (V) | (h) | (%) | (%) | trans) | isomer) |
| 1 | 15 | 60 | 10 | 72 | 100% | / | 32.9 | 74.3% |
| 2 | 3.5 | 30 | 10 | 72 | 100% | 87% | 182.9 | 82.9% |
| 3 | 3.5 | 40 | 10 | 72 | 100% | / | 97.1 | 76.4% |
| 4 | 3.5 | 30 | 10 | 48 | 98% | / | 152.2 | 80.7% |
| 5 | 3.5 | 60 | 10 | 6 | 100% | 95% | 50.6 | 74.1% |
| 6 | 3.5 | 60 | 5 | 6 | 100% | 94% | 61 | 74.8% |
| 7 | 3.5 | 50 | 5 | 6 | 100% | / | 101.7 | 78.9% |
| 8 | 2 | 60 | 5 | 6 | 100% | / | 33.9 | 73.6% |
| 9 | 2 | 50 | 10 | 24 | 98% | / | 88.5 | 79.4% |
| 10 | 2 | 40 | 10 | 24 | 98% | / | 41.6 | 75.7% |

### Example 3

### Preparation of methyl ((3S,4S)-1-benzyl-4-methylpiperidin-3-yl)carbamate (II-SS)

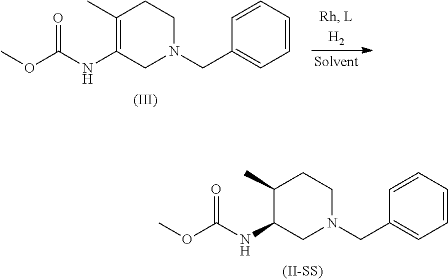

To a 250 mL pressure vessel were added (1-benzyl-4-methyl-1,2,5,6-tetrahydropydin-3-yl)carbamate (III) (1.0 g, 3.84 mmol), bis(1,5-cyclooctadiene)rhodium(I) trifluoromethanesulfonate (18.0 mg, 0.0384 mmol, CAS: 99326-34-8), (S)-1-[(Rp)-2-(di-tert-butylphosphino)ferrocenyl]ethylbis(2-methylphenyl)phosphine (22.0 mg, 0.0384 mmol, CAS: 849924-77-2). The solids were purged with nitrogen (5×5 bar) then trifluoroethanol was added (10 mL, 10 V). The solution was purged with nitrogen (5×5 bar) followed by hydrogen (3.5 bar). The reaction was heated to 30° C. maintaining the hydrogen pressure at 3.5 bar. After 72 h, the mixture was cooled and purged with nitrogen (5×5 bar). An aliquot was removed for HPLC analysis to confirm full reaction conversion. The mixture was concentrated to dryness and methyl-((3S,4S)-1-benzyl-4-methylpiperidin-3-yl)carbamate (II-SS) (990 mg, 87% yield, 82.9% e.e. and dr 182.9 chiral HPLC) was isolated as an oil.

Initial hydrogen pressure is 3.5 bar. As the reaction proceeds, pressure falls to 0 bar. The vessel is re-charged to 3.5 bar repeatedly until no further hydrogen consumption is observed (requiring approx. 5 re-charges).

### Example 4

### Preparation of methyl ((3R,4R)-1-benzyl-4-methylpiperidin-3-yl)carbamate (II-RR)

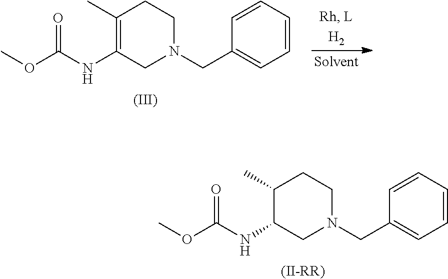

To a 15 mL pressure vessel were added (1-benzyl-4-methyl-1,2,5,6-tetrahydropyidin-3-yl)carbamate (III) (1.0 g, 3.84 mmol), bis(1,5-cyclooctadiene)rhodium(I) trifluoromethanesulfonate (18.0 mg, 0.0384 mmol,

CAS: 99326-34-8) and (R)-1-[(Sp)-2-(di-tert-butylphosphino)ferrocenyl]ethyl-bis (2-methylphenyl)phosphine (22.0 mg, 0.0384 mmol, CAS: 849924-76-1) in trifluoroethanol (5 mL, 5 V). The solution was purged with nitrogen (5×5 bar) followed by hydrogen (3.5 bar). The reaction was heated at 30° C. for 1 h then at 50° C. for 5 h, maintaining the hydrogen pressure at 3.5 bar. The mixture was cooled and purged with nitrogen (5×5 bar). An aliquot was removed for HPLC analysis to confirm full reaction conversion (97% cony.). The mixture was concentrated to dryness to provide methyl ((3R,4R)-1-benzyl-4-methylpiperidin-3-yl)carbamate (II-RR) (1.08 g, quant. yield, 82.3% e.e. and dr 101.7 chiral HPLC) as an oil.

Initial hydrogen pressure is 3.5 bar. As the reaction proceeds, pressure falls to 0 bar. The vessel is re-charged to 3.5 bar repeatedly until no further hydrogen consumption is observed (requiring approx. 3 re-charges).

### Example 5

### General preparation of 1-benzyl-N,4-dimethylpiperidin-3-amine (VII)

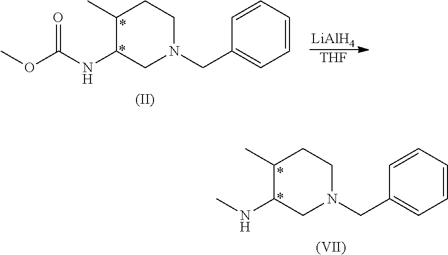

To a nitrogen-purged round-bottomed flask containing methyl (1-benzyl-4-methylpiperidin-3-yl)carbamate (II) (1 g, 4.28 mmol) in THF (10 mL) was added dropwise LiAlH₄ 1 M solution in THF (12.07 mL, 13.69 mmol) at 0° C. The reaction mixture was stirred at 0° C. for 5 min and was then heated at reflux for 2.5 h. After complete reaction, the mixture was cooled to 0-5° C. and water (2 mL) was slowly added over 5 min. The resulting suspension was filtered and the cake was washed with THF (4 mL). The combined filtrate and washings were concentrated to dryness to give 1-benzyl-N,4-dimethylpiperidin-3-amine (VII) as an oil.

### Example 6

### Preparation of (3S,4S)-1-benzyl-N,4-dimethylpiperidin-3-amine (VII-SS)

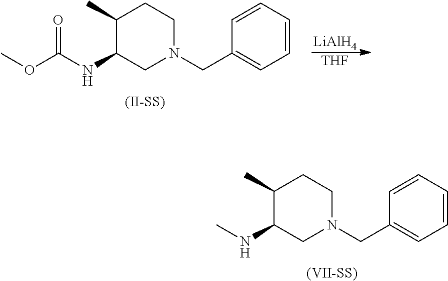

To a nitrogen-purged round-bottomed flask containing methyl ((3S,4S)-1-benzyl-4-methylpiperidin-3-yl)carbamate (II-SS) (0.99 g, 4.28 mmol) in THF (10 mL) was added dropwise LiAlH₄ 1 M solution in THF (12.07 mL, 13.69 mmol) at 0° C. The reaction mixture was stirred at 0° C. for 5 min and was then heated at reflux for 2.5 h. After complete reaction, the mixture was cooled to 0-5° C. and water (2 mL) was slowly added over 5 min. The resulting suspension was filtered and the cake was washed with THF (4 mL). The combined filtrate and washings were concentrated to dryness to give (3S,4S)-1-benzyl-N,4-dimethylpiperidin-3-amine (VII-SS) (0.512 g, crude, 59% yield) as an oil.

### Example 7

### Preparation of (3R,4R)-1-benzyl-N,4-dimethylpiperidin-3-amine (VII-RR)

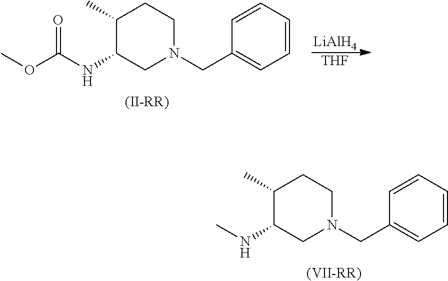

To a nitrogen-purged 100 mL round-bottomed flask containing methyl ((3R,4R)-1-benzyl-4-methylpiperidin-3-yl)carbamate (II-RR) (3.84 mmol) in THF (10 mL, 10 V) was added dropwise LiAlH₄ (1 M solution in THF, 12.29 mL, 12.29 mmol) at 0° C. The reaction mixture was stirred at 0° C. for 20 min and was then heated at reflux for 2.5 h. After complete reaction, the mixture was cooled to 0-5° C. and water (2 mL, 2 V) was slowly added over 5 min. The resulting suspension was filtered and the cake was washed with THF (4 mL). The combined filtrate and washings were concentrated to dryness to provide (3R,4R)-1-benzyl-N,4-dimethylpiperidin-3-amine (VII-RR) (0.625 g, 91.5% e.e. chiral GC) as an oil.

### Example 8

### General preparation of 1-benzyl-N,4-dimethylpiperidin-3-amine dihydrochloride (VIII)

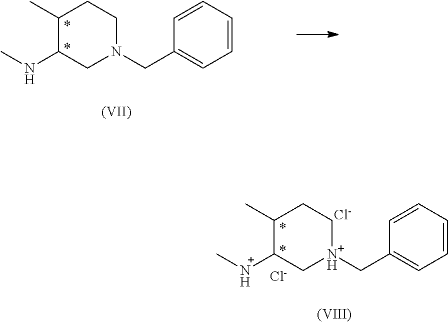

To a solution of 1-benzyl-N,4-dimethylpiperidin-3-amine (VII) (0.5 g, 2.34 mmol, in isopropanol (1.5 mL) was added a solution of 6 N HCl in isopropanol (1.25 mL, 3.2 eq). To the suspension formed was added heptane (1 mL) and the mixture was heated at reflux for 10 min. The suspension was stirred at RT for 30 min and the solid was filtered and washed with cold heptane (2×2 mL). The wet cake was dried at RT under vacuum to obtain 1-benzyl-N,4-dimethylpiperidin-3-amine dihydrochloride (VIII) as a pale solid.

### Example 9

### Preparation of (3S,4S)-1-benzyl-N,4-dimethylpiperidin-3-amine dihydrochloride (VIII-SS)

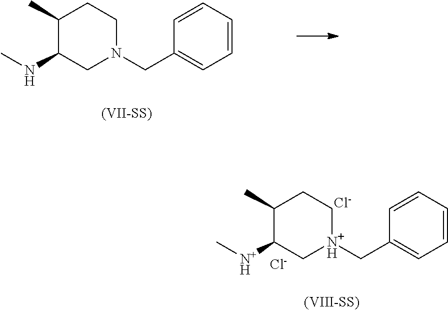

To a solution of (3S,4S)-1-benzyl-N,4-dimethylpiperidin-3-amine (VII-SS) (0.512 g, 2.34 mmol) in isopropanol (1.5 mL) was added a solution of 6 N HCl in isopropanol (1.25 mL, 3.2 eq). To the suspension formed was added heptane (1 mL) and the mixture was heated at reflux for 10 min. The suspension was stirred at RT for 30 min and the solid was filtered and washed with cold heptane (2×2 mL). The wet cake was dried at RT under vacuum to obtain (3S,4S)-1-benzyl-N,4-dimethylpiperidin-3-amine dihydrochloride (VIII-SS) (467 mg, 69% yield, 93.5% e.e. GC) as a pale solid.

### Example 10

### Preparation of (3R,4R)-1-benzyl-N,4-dimethylpiperidin-3-amine dihydrochloride (VIII-RR)

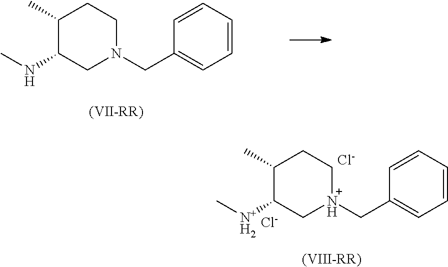

To a solution of (3R,4R)-1-benzyl-N,4-dimethylpiperidin-3-amine (VII-RR) (0.615 g, 2.82 mmol) in isopropanol (1.85 mL, 3 V) was added a solution of 6 N HCl in isopropanol (1.50 mL, 9.01 mmol, 3.2 eq). To the resulting suspension was added heptane (1 mL) and the mixture was heated at reflux temperature for 10 min. The suspension was stirred at RT for 30 min and the solid was filtered and washed with cold heptane (2×2 mL). The wet cake was dried at RT under vacuum to obtain (3R,4R)-1-benzyl-N,4-dimethylpiperidin-3-amine dihydrochloride (VIII-RR) (488 mg, 60% yield, 97.3% e.e. chiral GC) as a pale solid.

### Example 11

### General recrystallization of 1-benzyl-N,4-dimethylpiperidin-3-amine dihydrochloride (VIII) in Methanol

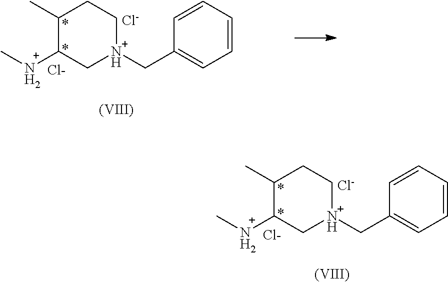

A suspension of 1-benzyl-N,4-dimethylpiperidin-3-amine dihydrochloride (VIII) (2.0 g) in MeOH (10 V) was heated at reflux until a solution was obtained. The mixture was cooled to RT and held for 12 h. The solid was filtered and washed with cold MeOH (2×1 V). The wet cake was dried at RT under vacuum to give the title salt (68% yield, 99% e.e.).

### Example 12

### General recrystallization of 1-benzyl-N,4-dimethylpiperidin-3-amine dihydrochloride (VIII) in THF/H₂O (5%)

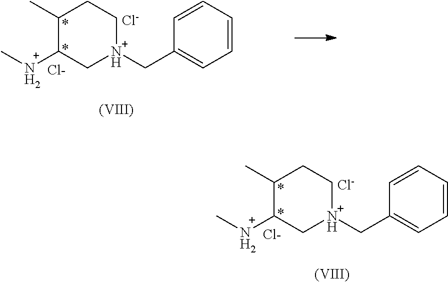

A suspension of 1-benzyl-N,4-dimethylpiperidin-3-amine dihydrochloride (VIII) (2.0 g) in THF/H2O(5%) (20 V) was heated at reflux until a solution was obtained. The mixture was cooled to RT and held for 12 h followed by 24 h at 0° C. The solid observed in the aqueous layer was filtered and washed with cold THF (2×1 V). The wet cake was dried at RT under vacuum to give the title salt (24% yield, 98% e.e.).

### Example 13

### Analytical Methods

Analytical method for determining the e.e. of the present invention. The method monitoring the result of example from 1 to 4 and the chiral purity of the compound of formula (II), via HPLC, chromatographic conditions:

Colum: Chiralcel OJ;

Temp. Colum: 250° C.;

Mobile Phase: Heptane/IPA 95:5;

Mode: Isocratic;

Flow: 0.5 mL/min;

UV Detector: 210 nm;

Injection Volume: 5 μL;

Analysis Time: 25 min;

Diluent: Hepatane/IPa 1/1;

### Retention Time:

Syn (S,S)=6.8 min;

Syn (R,R)=9.2 min;

Anti-(R,S) and (S,R)=12.3 and 19.0 min (specific enantiomers not known).

Analytical method for determining the e.e. of the present invention. The method monitoring the result of example from 5 to 12 and the chiral purity of the compound of formula (VII) and (VIII), via GC, chromatographic conditions:

Colum: Cyclosil-B;

Temp. Inj: 230° C.;

Temp. Det.: 250° C.;

Oven Temperature program:

start a 120° C.;

ramp at 3° C./min from 120° C. to 205° C.;

hold at 205° C. for 2 minute;

ramp at 10° C./min from 205° C. to 220° C.;

hold at 220° C. for 5 minutes.

Flow: 1.0 mL/min;

Split: 50:1

UV Detector: 260 nm;

Injection Volume: 1 μL;

Analysis Time: 37 min;

Diluent: dichloromethane;

### Retention Time:

Syn (S,S)=28 min;

Syn (R,R)=28.1 min.

1. A process for the preparation of a compound of formula (II) or a salt thereof: 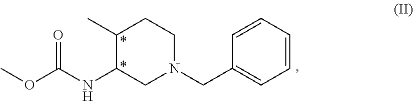 [SMILES: COC(=O)NC1CN(Cc2ccccc2)CCC1C]
wherein the asymmetric carbons marked with the symbol * have an optical configuration of (1) 3-R and 4-R or (2) 3-S and 4-S, or a mixture thereof, with the exclusion of the racemic mixture;
the process comprising asymmetrical hydrogenation of the compound of formula (III) or a salt thereof: 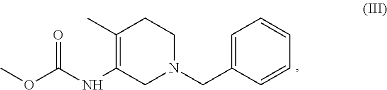 [SMILES: COC(=O)NC1=C(C)CCN(Cc2ccccc2)C1]
wherein said asymmetrical hydrogenation is carried out in a solvent and in presence of a Rh(I) complex and an optically active ferrocenyl phosphine, and the solvent is 2,2,2-trifluoroethanol or methanol.

2. The process according to the claim 1, wherein the compound of formula (II) or salt thereof has an enantiomeric excess higher than 67%, or of at least 70%.

3. The process according to claim 1, wherein the Rh(I) complex is a neutral complex of the general formula (IVa) or (IVb):
[RhLA]₂ (IVa) or
[RhL₂A] (IVb),
wherein L represents a C₄₋₁₂ diene or two C₂₋₁₂ alkene molecules, and A is chlorine, bromine, iodine, trifluoromethanesulfone, tetrafluoroboarte or acetylacetonate.

4. The process according to the claim 3, wherein L is norbornadiene or 1,5-cyclooctadiene.

5. The process according to claim 3, wherein A is trifluoromethansulfone.

6. The process according to claim 1, wherein the ferrocenyl phosphine has the following formula (V): 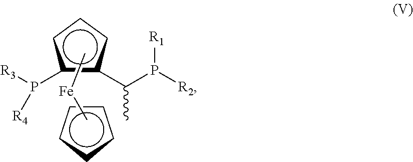
wherein R₁, R₂, R₃, and R₄ are independently selected from linear or branched C₁₋₅ alkyl, unsubstituted aryl, substituted aryl with a linear or branched C₁₋₅ alkyl group, or a cyclic C₅₋₆ alkyl;

7. The process according to the claim 6, wherein the ferrocenyl phosphine of formula (V) is (R)-1-[(S_{P})-2-(Di-tert-butylphosphino)ferrocenyl]ethylbis(2-methylphenyl) phosphine, having the formula (VI):

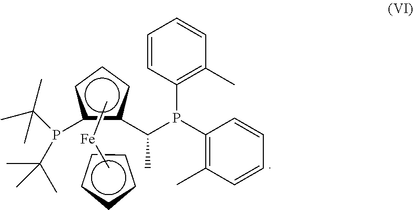

8. The process according to claim 1, wherein the asymmetrical hydrogenation is carried out at a temperature from 30° C. to 60° C. and the solvent is 2,2,2-trifluoroethanol, or is carried out at a temperature from 50° C. to 70° C. and the solvent is methanol.

9. The process according to claim 1, wherein the asymmetrical hydrogenation is carried out in 2,2,2-trifluoroethanol and the pressure is from 2 to 15 bar, or is carried in methanol and the pressure is from 10 to 20 bar.

10. The process according to claim 1, wherein the asymmetrical hydrogenation is carried out in from 5 to 10 volumes of 2,2,2-trifluoroethanol or from 10 to 20 volumes of methanol.

11. The process according to claim 1, wherein the compound of formula (II) or a salt thereof has the asymmetric carbons marked with the symbol * in the 3-R and 4-R optical configuration, has the formula (II-RR): 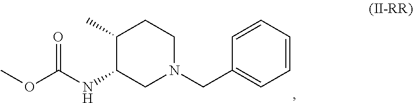 [SMILES: COC(=O)N[C@H]1CN(Cc2ccccc2)CC[C@H]1C]
and has an enantiomeric excess higher than 67%, or at least 70%.

12. The process according to claim 1, further comprising reducing the compound of formula (II) or a salt thereof to give the compound 1-benzyl-N,4-dimethylpiperidin-3-amine of formula (VII) or a salt thereof: 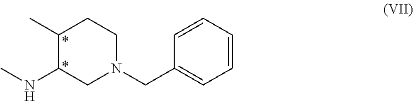 [SMILES: CNC1CN(Cc2ccccc2)CCC1C]
wherein the asymmetric carbons marked with the symbol * have an optical configuration of (1) 3-R and 4-R or (2) 3-S and 4-S, or mixture thereof, with the exclusion of the racemic mixture.

13. The process according to the claim 12, wherein the compounds of formula (II) and (VII) have the asymmetric carbons marked with the symbol * in the 3-R and 4-R optical configuration in an enantiomeric excess higher than 67%, or at least 70%, and the following formula: 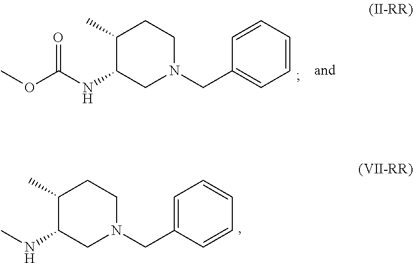 [SMILES: COC(=O)N[C@H]1CN(Cc2ccccc2)CC[C@H]1C ; CN[C@H]1CN(Cc2ccccc2)CC[C@H]1C]
respectively.

14. The process according to claim 13, further comprising converting the compound of formula (VII-RR) into the compound of formula (I): 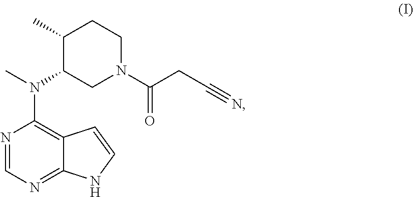 [SMILES: C[C@@H]1CCN(C(=O)CC#N)C[C@@H]1N(C)c1ncnc2[nH]ccc12]
having an enantiomeric excess higher than 67%, or at least 70%.

15. A process for the preparation of Tofacitinib having the following formula: 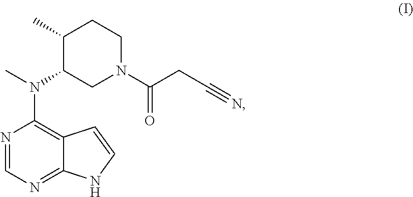 [SMILES: C[C@@H]1CCN(C(=O)CC#N)C[C@@H]1N(C)c1ncnc2[nH]ccc12]
or a salt thereof, and
having an enantiomeric excess higher than 67%, or at least 70%, the process comprising the following steps:
A) preparing the compound of formula (II-RR): 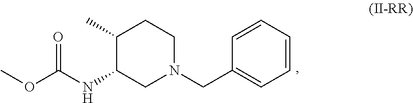 [SMILES: COC(=O)N[C@H]1CN(Cc2ccccc2)CC[C@H]1C]
having an enantiomeric excess higher than 67%, or at least 70%, according to the process of claim 11;
B) reducing the compound of formula (II-RR) obtained in the step A) to give the compound (3R,4R)-1-benzyl-N,4-dimethylpiperidin-3-amine of formula (VII-RR): 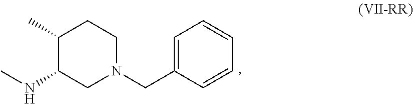 [SMILES: CN[C@H]1CN(Cc2ccccc2)CC[C@H]1C]
or a salt thereof,
having an enantiomeric excess higher than 67%, or at least 70%; and
C) converting the compound of formula (VII-RR) obtained in the step B) to Tofacitinib of formula (I) or a salt thereof, having an enantiomeric excess higher than 67%, or at least 70%.

16. A method of preparing the compound of formula (II): 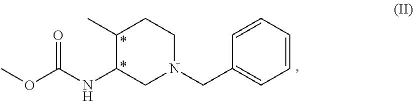 [SMILES: COC(=O)NC1CN(Cc2ccccc2)CCC1C]
or a salt thereof
wherein the asymmetric carbons marked with the symbol * have an optical configuration of (1) 3-R and 4-R or (2) 3-S and 4-S, or mixture thereof, with the exclusion of the racemic mixture, the method comprising asymmetric hydrogenation of the compound of formula (III) 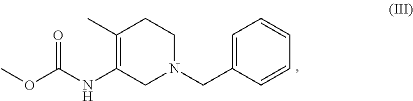 [SMILES: COC(=O)NC1=C(C)CCN(Cc2ccccc2)C1]
or a salt thereof, in 2,2,2-trifluoroethanol.

17. The process according to claim 4, wherein A is trifluoromethansulfone.
